(19) United States
(12) Patent Application Publication
(10) Pub. No.: US 2018/0085081 A1
(43) Pub. Date: Mar. 29, 2018

(54) RADIOGRAPHIC IMAGING APPARATUS

(71) Applicant: SHIMADZU CORPORATION, KYOTO (JP)

(72) Inventor: MASAHIRO TANAKA, KYOTO (JP)

(73) Assignee: SHIMADZU CORPORATION, KYOTO (JP)

(21) Appl. No.: 15/273,937

(22) Filed: Sep. 23, 2016

(51) Int. Cl. A61B 6/00; H05G 1/56; H05G 1/46; A61B 6/06

(52) U.S. Cl. CPC A61B 6/54; A61B 6/06; H05G 1/46; H05G 1/56

(57) ABSTRACT

A radiation apparatus conducts a subtraction imaging process where a holder 25 begins to shift when a radiation output ends relative to a first radiation in association with the subtraction imaging and then, once the filter is switched by shifting the holder 25 and when switching of the filter is actually measured, the X-ray tube outputs the radiation in association with a second imaging. With this structure, dual filter is not required to be switched and the imaging can be conducted immediately after an operator so instructs regardless the dual filter phase. The second imaging can be performed immediately after confirming the dual filter is switched in fact.

### CROSS REFERENCE TO RELATED APPLICATIONS

This application relates to, but does not claim priority from, Japanese Ser. No. JP 2014-045045 filed Mar. 7, 2014 which published on Sep. 28, 2015, the entire contents of which are incorporated herein by reference.

### FIGURE SELECTED FOR PUBLICATION

FIG. 1

### BACKGROUND OF THE INVENTION

### Field of the Invention

The present invention relates to a radiographic imaging apparatus for imaging a subject using a radiation and particularly relates to a radiographic imaging apparatus for a subtraction imaging and related systems and methods.

### Description of the Related Art

In a medical facility, a radiographic imaging apparatus is equipped to perform an imaging of a subject M by irradiating a radiation (e.g., Patent Document 1.) Referring now to FIG. 16, such a conventional radiographic imaging apparatus comprises a radiation source 53 to irradiate a radiation and a flat panel detector (FPD) 54 that detects the radiation. A table 52, on which the subject M can be laid, is equipped between the radiation source 53 and the FPD 54.

Some of such radiographic apparatus may acquire a subtraction image in which the soft tissues and the bone region of the subject are enhanced by obtaining the difference between 2 images obtained by conducting two kinds of imaging. The inventor sets forth the operation when a subtraction image is imaged using a conventional radiographic apparatus.

According to the conventional radiation tomographic apparatus, an imaging using a high voltage radiation source 53 and an imaging using a low voltage therefor are individually conducted. When such two images are compared, the aspects incorporating the subject's image are different. Specifically, the contrast difference between soft tissues and bones of the subject incorporated in the image under a high voltage is very different from the contrast difference between soft tissues and bones of the subject incorporated in the image under a low voltage. This creates great difficulty. Therefore, if the image under the high voltage condition is subtracted from the image under the low voltage condition, both images are not only just canceled, but also either soft tissues or bones can be enhanced. The conventional radiographic imaging apparatus 51 acquires a subtraction image, in which the soft tissues and the bone region of the subject are enhanced, utilizing such fact.

The conventional radiation imaging apparatus 51 comprises a filter 53f to change the radiation quality of the radiation source 53 (refer to FIG. 16.) Such filter 53f consists of a gadolinium filter for the high voltage irradiation and a copper filter for the low voltage irradiation. The gadolinium filter and the copper filter can be switched by controlling the drive element of the filter 53f (e.g., Patent Document 1.) If such filter 53f is installed, the difference between the image under the high voltage condition and the image under the low voltage condition can appear further clearly.

Specifically, and unfortunately, such imaging for the subtraction imaging must be conducted twice under the different conditions. At that time, if the subject moves between the first imaging and the second imaging, the clear subtraction image cannot be obtained. If the positional deviation between two subject's images takes place, the process in which the second subject's image is subtracted from a part of the first image having nothing incorporated in the profile area of the subject is executed, so that the subtraction image is disrupted.

An increase of the continuous shooting speed is effective on preventing the disruption of the subtraction image. The faster the continuous shooting speed is, the smaller the subject's positional deviation between the two images is.

It is deemed that the switching speed of the filter should be much faster to increase the continuous shooting rate. In such way, the structure in which the switching rate of the filter is increased to improve the continuous shooting speed is already proposed. Referring to FIG. 17, such device provides the disk consisting of the combination of the semicircle high voltage imaging filter (high voltage filter) and the similar semicircle low voltage filter (low voltage filter.) The filter can be switched in a high speed by rotating such disks (refer to e.g., Patent Document 1.)

### PRIOR ART RELATED ART DOCUMENTS

### Patent Document

Patent Document: Laid Open JP 2009-293

### ASPECTS AND SUMMARY OF THE INVENTION

### Objects to be Solved

However, there are following problems remained in the conventional system. Specifically, according to the conventional system, the apparatus control is difficult on imaging a subtraction image.

According to the conventional system, the rotation rate of the filter determines the continuous shooting speed. Accordingly, if the condition of the subtraction imaging, particularly the continuous shooting speed, is desired to be changed, first of all, the rotation rate of the filter must be changed. Since the filter is rotating with a constant force, the change of such rotation rate takes a time in some extent to settle down. According to the conventional system, the imaging is suspended until the rotation rate of the filter becomes as set. Such defect takes place even when the suspended filter begins to rotate at the beginning of subtraction imaging.

In addition, according to the conventional system, the filter phase determines the timing of imaging. Even when the operator instructs the apparatus to initiate an imaging, the high power imaging may not be available immediately. Because no guarantee is provided whether the filter is switched to the high power condition or not, when the instruction as to the initiation of imaging is given. The imaging becomes only available to begin after the filter is switched to the high power condition. The operator may feel as if a slow operation because the imaging does not begin even the imaging initiation is instructed.

Such inconvenience relative to a use of the apparatus may control an imaging limitation due to the rotating filter. Such conventional system is not desirable because the imaging per se depends on the filter's condition. Principally, the filter per se must be configured out to be able to switch in association with the imaging.

The present invention is motivated under such circumstances and the purpose thereof is to provide a radiation apparatus having a radiation filter switchable quickly in association with the imaging condition so that the subtraction imaging can be further assuredly executed.

### Means for Solving the Problem

The present invention comprises the following structures to solve the above problem.

Specifically, a radiographic imaging apparatus according to the aspect of the present invention is the radiographic imaging apparatus that images a subtraction image by imaging twice using respectively different conditions comprises; a radiation source to irradiate a radiation, a radiation source control means so as to irradiate alternately the radiation in association with a high voltage and the radiation in association with a low voltage, a high voltage filter that allows the radiation in association with the high voltage, a low voltage filter that allows the radiation in association with the low voltage, a holder supporting the high voltage filter and the low voltage filter, a holder shifting means that switches a dual (combination) filter, through which the radiation passes, between the high voltage filter and the low voltage filter by shifting the holder, a holder shifting control means to control said holder shifting means, a detection means that detects the dual filter, through which the radiation passes, switched to which either the high voltage filter or the low voltage filter, wherein the holder shifting means initiates to shift the holder from the time when the radiation output in association with the first imaging ends by receiving the radiation output signal, which indicates whether the radiation is being output or not, from the radiation source control means, and the detection means detects that the dual filter is switched following the holder's shift and sends the signal indicating the fact to the radiation control means, and the radiation source control means allows the radiation source to output the radiation in association with the second imaging following receiving the signal indicating that the dual filter has been shifted.

### Action and Effect

The radiation apparatus according to the present invention comprises a radiation filter switchable quickly in association with the imaging condition so that the subtraction imaging can be further assuredly executed. Specifically, according to the structure of the present invention, the holder begins to shift since when the radiation output ends relative to the first radiation in association with the subtraction imaging and then once the filter is switched by shifting the holder and when switching of the filter is actually measured, the radiation source outputs the radiation in association with the second imaging. According to the present invention, the filter can be changed to the suitable filter for imaging prior to the first imaging differently from the conventional aspect. Therefore, according to the aspect of the present invention, the imaging can be conducted right after the operator instructs regardless the filter phase.

Further, according to the structure of the present invention, the second imaging can be performed right after it is confirmed that the filter is switched in fact. Therefore, according to the structure of the present invention, the change of the continuous imaging rate can be achieved further quickly. For example, according to the aspect of the present invention, when the continuous imaging rate is changed so as to be slower, the first imaging is first of all performed and then the second imaging can be delayed to begin as long as needed. Referring to FIG. 17, if the high voltage filter and the low voltage filter are configured to rotate, the rotation rate must be changed, so that even first imaging is not operable until such rate becomes stable. According to the aspect of the present invention, such limitation would not take place.

Further, the above image processing device further preferably comprises one subtraction imaging filter that is arranged in the shifting direction of the high voltage filter and the low voltage filter.

### Action and Effect

The above system illustrates further specifically a radiographic apparatus of the present invention. If just one subtraction imaging filter is arranged and structured in the shifting direction of the high voltage filter and the low voltage filter, switching filter between the high voltage filter and the low voltage filter can be performed by a slight shift of the holder.

Further, according to the above image processing device, it is further preferable that:

(A) a detection means comprises a light emission means that is in-place as if sandwiching the holder and a light detection means that detects the light, and also

(B1) the high voltage filter switching confirmation window through which the light passes only in the positional relationship between the holder, in the state in which the filter is changed to the high voltage filter, and the radiation source is installed to the holder.

Further, according to the above image processing device, it is further preferable that:

(A) a detection means comprises a light emission means that is in-place as if sandwiching the holder and a light detection means that detects the light, and also

(B2) the low voltage filter switching confirmation window through which the light passes only in the positional relationship between the holder, in the state in which the filter is changed to the low voltage filter, and the radiation source is installed to the holder.

### Action and Effect

The above system illustrates further specifically the present invention. If the high voltage filter switching is confirmed through the high voltage filter switching confirmation window, the timing at which the filter is switched can be more assuredly figured out. Such circumstance is also applicable to the low voltage filter.

### EFFECT OF THE INVENTION

According to the system of the present invention, the holder begins to shift since when the radiation output ends relative to the first radiation in association with the subtraction imaging and then once the filter is switched by shifting the holder and when switching of the filter is actually measured, the radiation source outputs the radiation relative to the second imaging. According to such structure, the filter is not required to be switched always and alternately so that the imaging can be conducted right after the operator instructs regardless the filter phase. Further, according to the structure of the present invention, the second imaging can be performed right after it is confirmed that the filter is switched in fact. According to the structure of the present invention, the change of the continuous imaging speed can be achieved further quickly.

The above and other aspects, features and advantages of the present invention will become apparent from the following description read in conjunction with the accompanying drawings, in which like reference numerals designate the same elements.

### BRIEF DESCRIPTION OF THE DRAWINGS

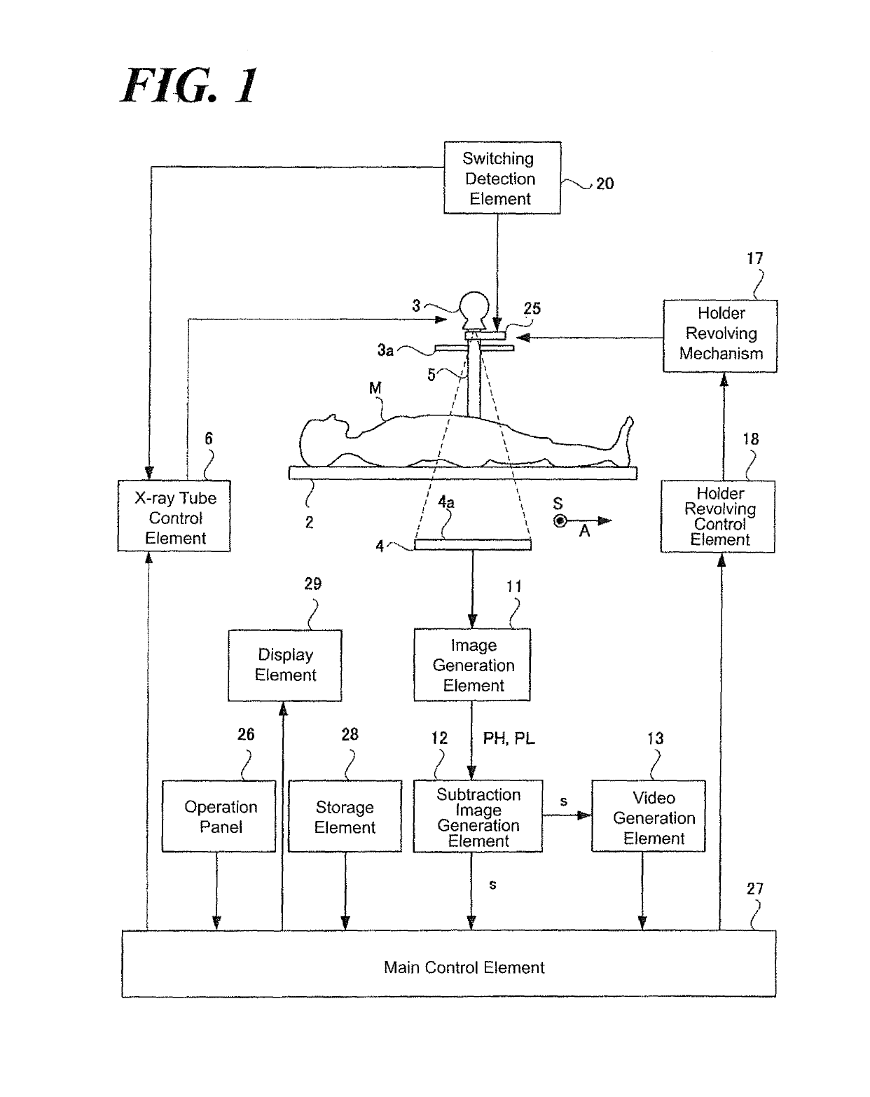

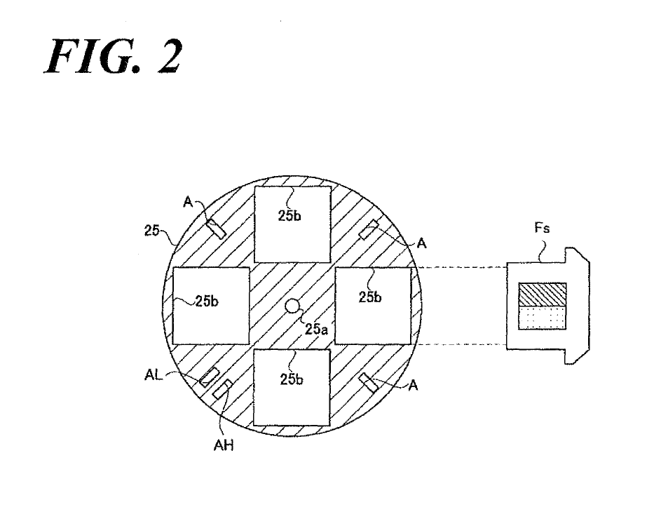

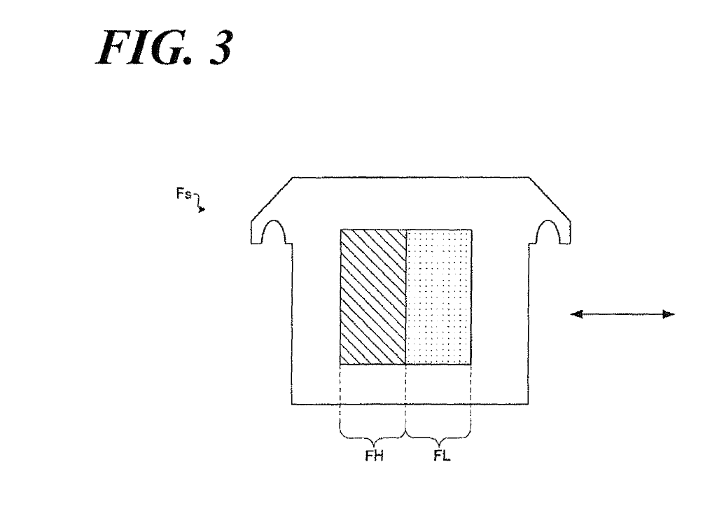

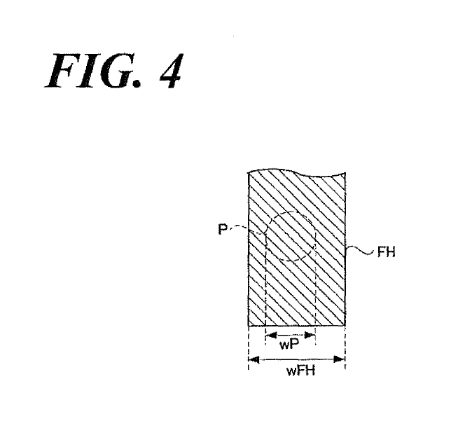

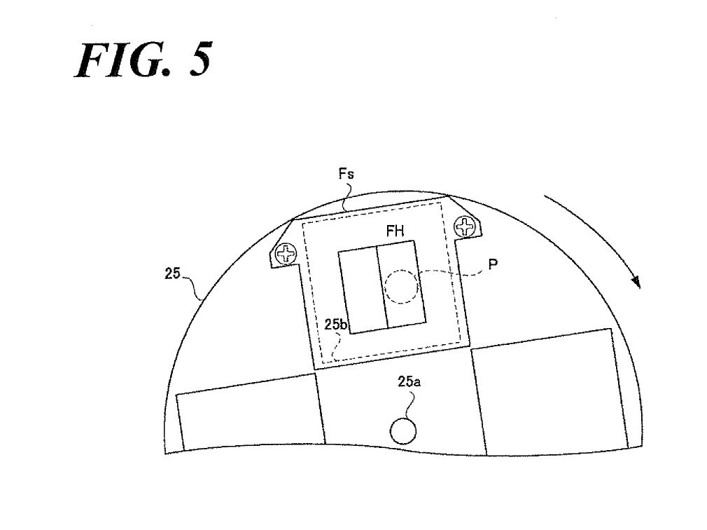

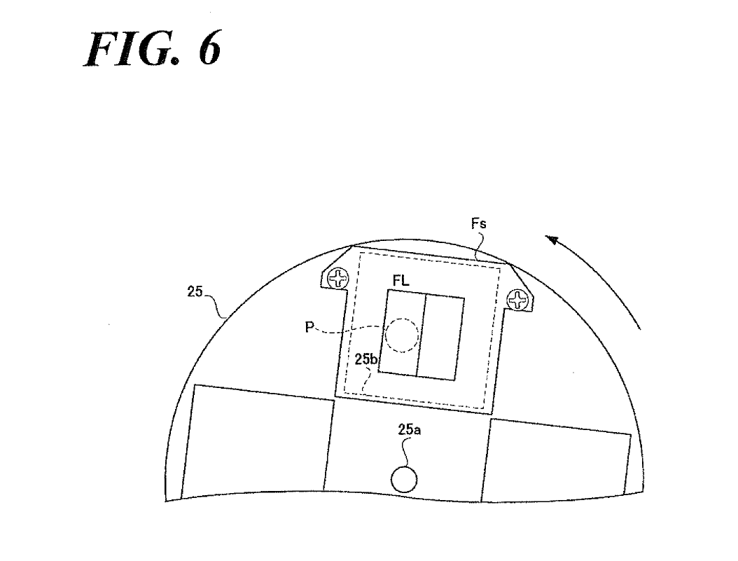

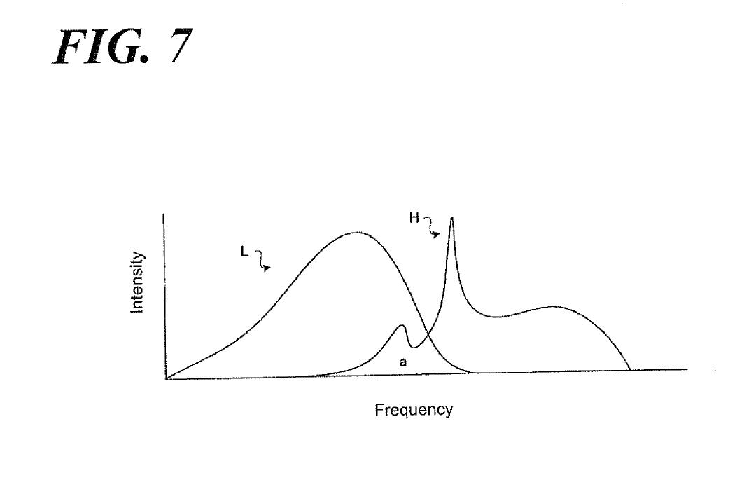

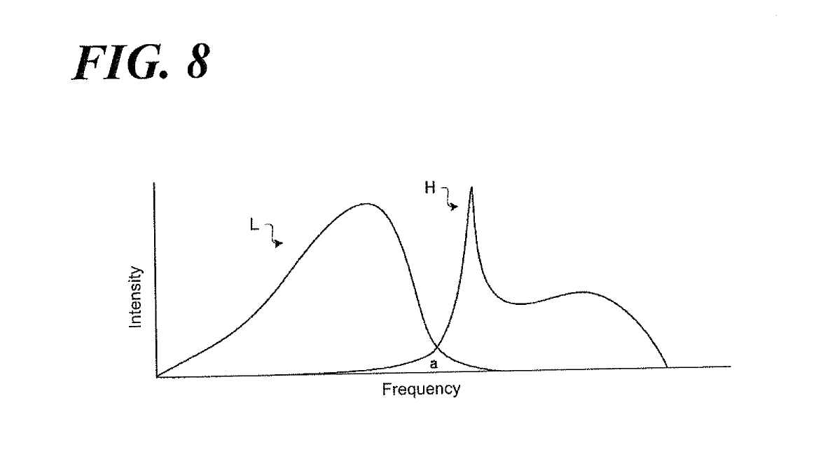

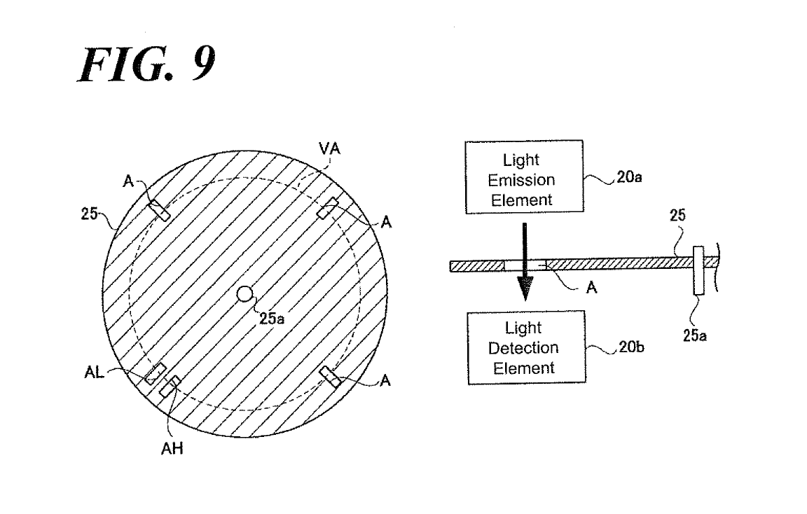

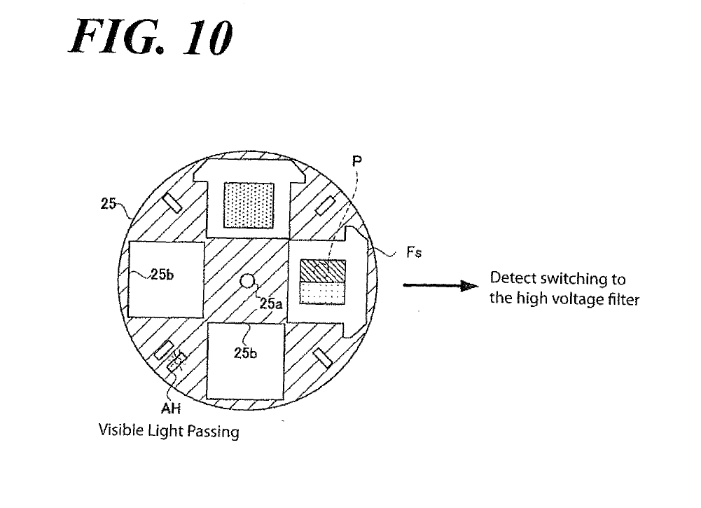

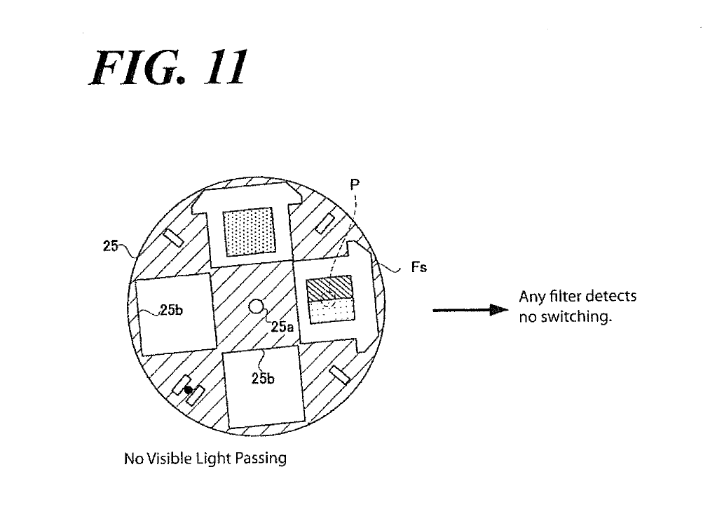

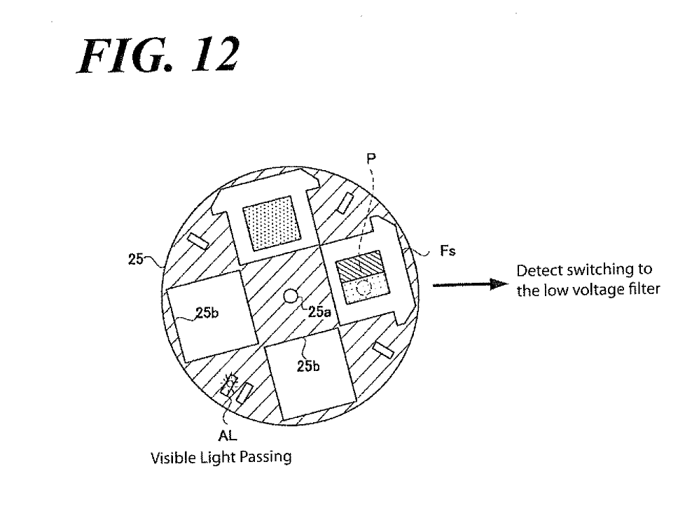

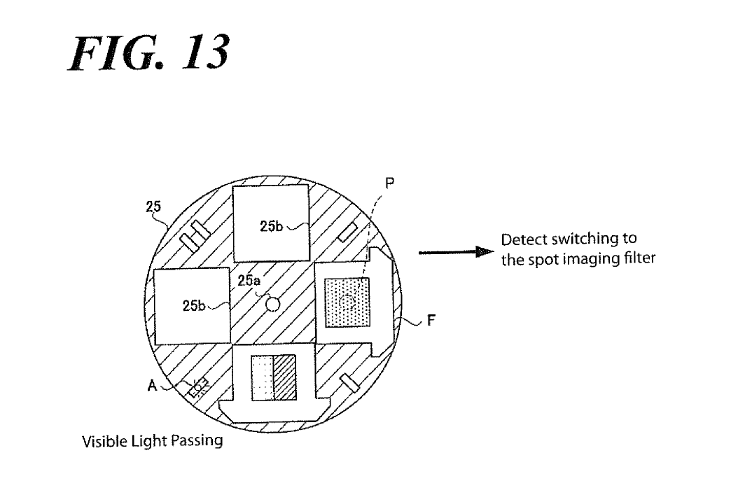

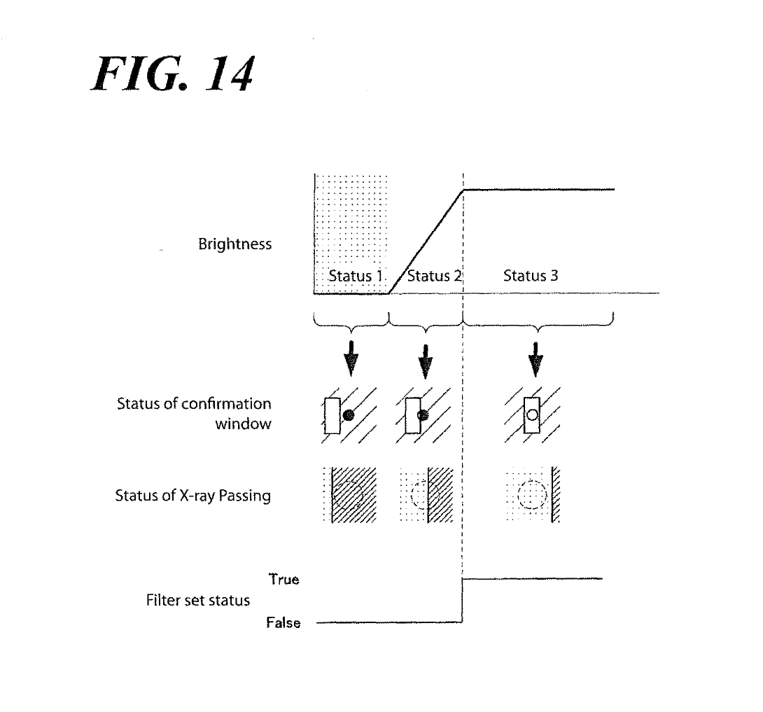

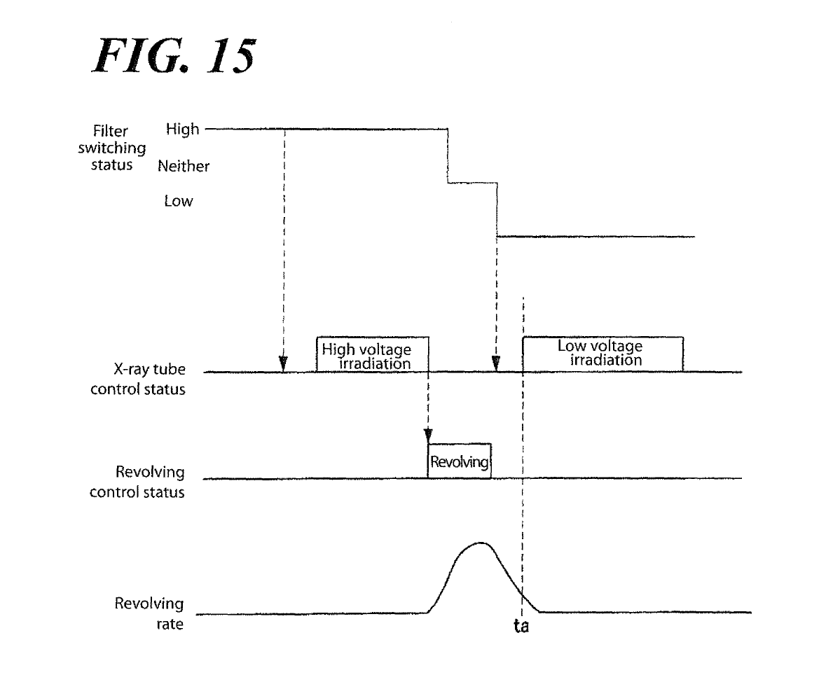

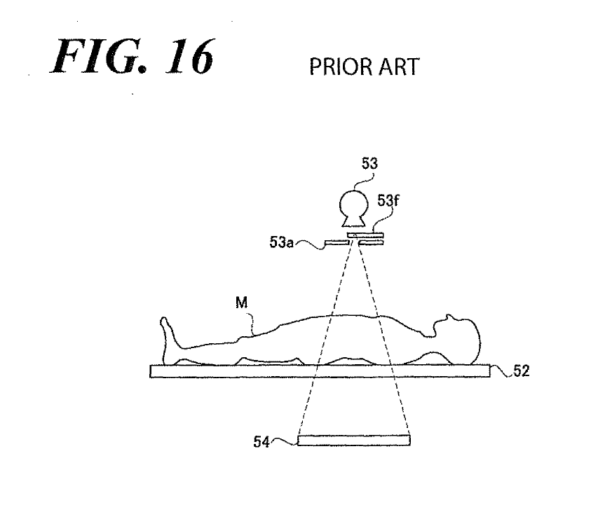

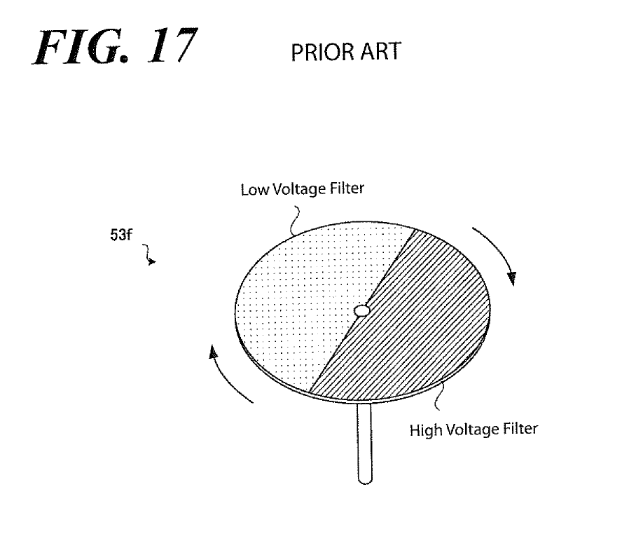

FIG. 1 is a functional block diagram illustrating the entire structure of an X-ray imaging device according to the Embodiment 1.

FIG. 2 is a plan view illustrating a holder according to the Embodiment 1.

FIG. 3 is a plan view illustrating a structure of a dual filter for the subtraction imaging according to the Embodiment 1.

FIG. 4 is a plan view illustrating a structure of a dual filter according to the Embodiment 1.

FIG. 5 is a schematic diagram illustrating a switching of the dual filter according to the Embodiment 1.

FIG. 6 is a schematic diagram illustrating a switching of the dual filter according to the Embodiment 1.

FIG. 7 is a schematic diagram illustrating a function of the dual filter according to the Embodiment 1.

FIG. 8 is a schematic diagram illustrating a function of the dual filter according to the Embodiment 1.

FIG. 9 is a schematic diagram illustrating a mechanism to detect switching of the dual filter according to the Embodiment 1.

FIG. 10 is a schematic diagram illustrating an aspect of detecting switching of the dual filter according to the Embodiment 1.

FIG. 11 is a schematic diagram illustrating an aspect of detecting switching of the dual filter according to the Embodiment 1.

FIG. 12 is a schematic diagram illustrating an aspect of detecting switching of the dual filter according to the Embodiment 1.

FIG. 13 is a schematic diagram illustrating an aspect of detecting switching of the single (filter according to the Embodiment 1.

FIG. 14 is a schematic diagram illustrating an aspect of detecting switching of the dual filter according to the Embodiment 1.

FIG. 15 is a functional block diagram illustrating a subtraction imaging operation according to the Embodiment 1.

FIG. 16 is a schematic diagram illustrating a conventional radiographic apparatus.

FIG. 17 is a schematic diagram illustrating a structure of a dual filter for the subtraction imaging according to the conventional system.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

### EMBODIMENT 1

### Entire System of the X-Ray Radiographic Apparatus

First, the inventor illustrates the system of the X-ray radiographic apparatus according to the aspect of the Embodiment 1. Referring to FIG. 1, an X-ray radiographic apparatus 1 comprises a table 2 on which a subject M in the supine position is laid, an X-ray tube 3 to irradiate an X-ray is mounted above the table 2 and a FPD 4 mounted under the table 2 to detect the X-ray. The FPD (flat panel detector) 4 has a rectangular shape with 4 sides along with either the axis direction A of body or the side direction S of body of the subject M. Further, the X-ray tube 3 irradiates the X-ray quadrangular pyramid beam to the FPD 4. The FPD 4 receives the X-ray on the whole surface thereof. The post 5 is extending from the underside (the other side) of the table 2 to the upper side (one side) of the table 2 and supports the X-ray tube 3. The X-ray tube 3 corresponds to the radiation source of the present invention. A radiographic imaging apparatus 1 according to the present invention is the radiographic imaging apparatus that images a subtraction image by imaging twice using respectively different conditions. Further, an X-ray corresponds to the radiation of the present invention.

A X-ray tube control element 6 (refer to FIG. 1) is installed in order to control the X-ray tube 3 with the predetermined tube electric current and the electric voltage and the pulse width. Once the X-ray is irradiated from the X-ray tube 3 under control by the X-ray tube control element 6, the X-ray transmits the subject M and is incident to the detection face of the FPD 4a. The FPD contains the florescence substance photosensitive to the X-ray so that the fluoroscopic image of the subject M is developed on the florescence substance and the light of the florescence substance is converted to an electric signal so as to provide the digital image. The X-ray tube control element 6 uses two modes, a high voltage mode to irradiate X-ray with a high tube voltage and a low voltage mode to irradiate X-ray with a low tube voltage with a high voltage, separately to perform a subtraction imaging. Such X-ray tube control element 6 controls the X-ray tube 3 to irradiate the X-ray alternately in association with a high voltage and the X-ray in association with the low voltage by using two modes separately. The X-ray tube 6 corresponds to the radiation source control means of the present invention.

### Dual Filter F

FIG. 2 is illustrating the structure of a holder 25 in association with the dual filter attached to a collimator 3a limiting the expansion of the X-ray irradiated from the X-ray tube 3. The holder 25 holds the dual filter that transmits the X-ray. The holder 25 is a disk-like member spreading on the plane perpendicular to the X-ray incident direction. Referring to FIG. 1, the holder 25 is in-place in the position sandwiched in between the X-ray tube 3 and the collimator 3a. Accordingly, the X-ray beam irradiated from the X-ray tube 3 passes through the holder 25 and reaches to the collimator 3a.

The holder 25 is revolvable relative to the X-ray tube 3. Specifically, a central axis 25a extending to the X-ray beam incident direction is installed to the center of the holder 25 and the holder 25 can revolve with freedom. A revolving drive of the holder 25 is executed by a revolving mechanism 17. The revolving control element 18 is installed to control the revolving mechanism 17. The holder revolving mechanism 17 corresponds to the holder shifting means of the present invention and the holder revolving control element 18 corresponds to the holder shifting revolving control means of the present invention.

The holder 25 includes a plurality of dual and/or single filters that block the part of X-ray. The holder 25 includes a plurality of rectangular holes 25b that penetrate the holder 25 to the X-ray beam incident direction. Accordingly, the holes 25b penetrate the holder 25 to the center axis C direction. The holes 25b are installed as surrounding the center axis 25s of the holder 25. Referring to FIG. 2, four holes 25b are installed, but the number of the holes 25b can be changed without limitation. The holder 25b is a penetrating hole installed to the holder 25 so that the X-ray beam can pass through. A different kind of dual filter Fs can be set or no dual filter Fs can be set. The holder 25 supports a variety of filters F, Fs including the dual filter Fs for the subtraction imaging comprising the high voltage filter FH and the low voltage filter FL.

The inventor sets forth the aspect of that the holder 25 holds the dual filter Fs. The dual filter Fs is fixed to the holder 25 so as to clog the holes 25b. Referring to FIG. 2, the aspect of that the dual filter Fs fixed to the holder 25 is removed is illustrated. The dual filter Fs is a plate member that is thin in the incident direction of the X-ray beam. The purpose of the dual filter Fs is to change the line quality of the X-ray beam to the desired line quality. Specifically, when the X-ray beam irradiated from the X-ray tube 3 passes the dual filter Fs, parts of the X-ray beam are absorbed so that such part of X-ray beam cannot reach to the subject M.

The holder 25 is revolved so that the kind of the dual filter Fs passing the X-ray beam can be changed. The holder 25 is revolved so that the position of the holder 25, through which the X-ray beam irradiated from the X-ray tube 3 passes, can be changed. According to such operation, the line quality of the X-ray beam incident to the subject M can be the desired line quality thereof. Because the dual filter Fs stored in the holder 25 is also revolved followingly. In addition, the holder 25 is revolved and thereby the X-ray beam passes the hole 25b without the embedded dual filter Fs so that the X-ray beam cannot pass through any dual filter Fs.

### Dual Filter Fs for the Subtraction Imaging

The inventor sets forth dual filter Fs for the subtraction imaging, which are installed to the holder 25. A half of the center portion of the dual filter Fs is an dual filter (high voltage filter FH) allowing the irradiated X-ray to pass through when the X-ray tube 3 is controlled under the high voltage mode. The other half of the center portion of the dual filter Fs is a dual filter (low voltage filter FL) allowing the irradiated X-ray to pass through when the X-ray tube 3 is controlled under the low voltage mode. Other than the above, the dual filter Fs includes an outer frame to support the high voltage filter FH and the low voltage filter FL.

The inventor sets forth an arrangement of the high voltage filter FH and the low voltage filter FL relative to the dual filter Fs. The high voltage filter FH and the low voltage filter FL are in-place to be adjacent each other with no space in-between, and are aligned in the direction indicated by the arrow referring to FIG. 3. The direction indicated by the arrow is the direction in which the dual filter Fs shifts by revolving the holder 25 when the dual filter Fs is fixed to the holder 25.

Therefore, if the holder 25 is revolved slightly under the state in which the high voltage filter FH is set to the X-ray tube 3, the low voltage filter FL becomes to be set to the X-ray tube 3 at this time. Specifically, a dual filter Fs, through which the X-ray passes, can be switched between the high voltage filter FH and the low voltage filter LH by shifting the holder 25 relative to the X-ray tube 3. A shifting of the holder 25 is executed by the holder revolving mechanism 17. In such way, the high voltage filter FH and the low voltage filter FL are arranged in the in revolving direction of the holder, so that one dual filter for the subtraction imaging is structured.

In addition, the revolving direction of the holder 25 relative to the high voltage filter FH and the low voltage filter FL can vary in some range. For example, referring to FIG. 4, the width wFH of the high voltage filter FH relative to the revolving direction of the holder 25 is set as wider than the width wP relative to the same direction of the passing region P that is the region of the high voltage filter FH through which the X-ray irradiated from the X-ray tube 3 passes. Accordingly, the state in which the dual filter for the imaging is switched to the high voltage filter FH does not mean that the holder 25 is in strictly predetermined angle and rather than, the revolving angle of the holder 25 is in the predetermined range. Nevertheless, when the dual filter is switched, the revolving control of the holder 25 set as the revolving angle of the holder 25 is the central value of the predetermined range. Such circumstance is also applicable to the low voltage filter FL.

A switching detection element 20 that structurally detects to which the dual filter is switched either the high voltage filter or the low voltage filter. The switching detection element 20 corresponds to the detection means of the present invention. The specific structure thereof is set forth later.

Referring to FIG. 5, the aspect in which the high voltage filter FH of the dual filter Fs is set to the X-ray tube 3 is illustrated. The passing region P of the X-ray is the pathway through which the X-ray irradiated from the X-ray tube 3 passes. According to the structure referring to FIG. 5. the X-ray irradiated from the X-ray tube 3 is directed to the collimator 3a side passing through a part of the high voltage filter FH of the dual filter Fs. At this time, the hole 25b is installed to the holder 25 so that the X-ray cannot be incident in the member structuring the holder 25.

Referring to FIG. 6, the aspect in which the low voltage filter FL of the dual filter Fs is set to the X-ray tube 3 is illustrated. The passing region P of the X-ray is the pathway through which the X-ray irradiated from the X-ray tube 3, which is directing to the subject, passes. According to the structure referring to FIG. 6, the X-ray irradiated from the X-ray tube 3 is directed to the collimator 3a side passing through a part of the low voltage filter FL of the dual filter Fs. At this time, the holes 25b are installed to the holder 25 so that the X-ray cannot be incident in the member structuring the holder 25.

The inventor sets forth a change of the region set in the X-ray tube 3. The holder 25 in the state referring to FIG. 5 is revolved from the low voltage filter FL toward the high voltage filter FH to be in the state referring to FIG. 6 so that the state in which the high voltage filter FH is set to the X-ray tube 3 can be changed to the state in which the low voltage filter FH is set thereto.

Reversely the holder 25 in the state referring to FIG. 6 is revolved from the high voltage filter FH toward the low voltage filter FL to be in the state referring to FIG. 5 so that the state in which the low voltage filter FL is set to the X-ray tube 3 can be changed to the state in which the high voltage filter FH is set thereto. Further, the positional relationship between the center axis 25a and the passing region P will not change due to the revolving holder 25.

In such way, when imaging the subtraction image, the holder revolving mechanism 17 revolves the holder 25 so that the high voltage filter FH of the dual filter Fs can be shifted to the position at which the X-ray passes through when the X-ray tube 3 is in a high voltage and the low voltage filter FL of the dual filter Fs can be shifted to the position at which the X-ray passes through when the X-ray tube is in a low voltage The holder revolving mechanism 17 switches alternately the filter, which the X-ray passes trough, by a revolving control element 18 in between the high voltage filter FH and the low voltage filter FL in accordance with that the X-ray tube 3 control element 6 radiates alternately the X-ray due to the high voltage and the X-ray due to the low voltage.

The inventor sets forth the reason why the dual filter Fs for subtraction are structured with two filters FH, FL. The dual filter Fs are installed in order to acquire a subtraction image s using the two images based on the different outputs from the X-ray tube 3. The subtraction image s is generated by subtracting an image acquired based on the low voltage control relative to the X-ray tube 3 from an image acquired based on the high voltage control relative to the X-ray tube 3. When the two original images for the subtraction image s are compared, darkness of the bone image of the subject M is different from darkness of the soft tissue image of the subject M.

Given two images, in which darkness of the soft tissue image is the same as darkness of the bone image, are subject to a subtraction process, both subject's images incorporated in the images are simply canceled each other and just removed. However, given two images, in which darkness of the soft tissue image is different from darkness of the bone image, are subject to a subtraction process, for example, phenomenon, as cancellation of both images does not take place strongly in the parts incorporating the soft tissue in the image and in contrast cancellation of both images take place strongly in the parts incorporating the bone in the image, can be observed. According to such example, the subtraction image in which the soft tissue of the subject M is more emphasized than the bone by subtracting two images can be obtained. A coefficient is modified on a subtraction process so that the bone of the subject M can be emphasized.

Darkness of the bone image of the subject M must be absolutely different from darkness of the soft tissue of the subject M between two acquired images so that a subtraction image s having high visibility can be acquired. Such difference between darkness of each image is based on the different quality of the X-ray irradiated when the two images are imaged. Given the X-rays having the same line quality are irradiated to image two images, darkness of the bone image and the soft tissue image between two images are getting similar. Even if the subtraction is taken between such two images, the soft tissue image of the subject M cannot be emphasize because both subject's images incorporated in the images are just canceled.

Referring to FIG. 7, X-ray spectra are illustrated when a subtraction image s is acquired without dual filter Fs. Referring to FIG. 7, H represents the spectrum of the X-ray irradiated from the X-ray tube 3 having high voltage and L represents the spectrum of the X-ray irradiated from the X-ray tube 3 having low voltage. Referring to FIG. 7, each spectrum has a different frequency distribution but it is found that a common area a in which each spectrum is partially overlapped exists. Such common area a implies that each X-ray having the same line quality is included during two X-ray radiations It is desirable that such common area a is small as much as possible so that the quality of the X-ray between when the X-ray tube 3 is in high voltage and in low voltage can be different absolutely.

Then, the dual filter Fs is applied to acquire the subtraction image s. Specifically, when the X-ray tube 3 is in high voltage, the X-ray passes through the high voltage filter FH of the dual filter Fs. The high voltage filter FH cuts the low frequency part of the X-ray under the high voltage. In addition, when the X-ray tube 3 is in the low voltage, the X-ray passes through the low voltage filter FL of the dual filter Fs. The low voltage filter FL cuts the high frequency part of the X-ray under the low voltage.

The dual filter Fs become operative so that the spectra of X-ray irradiated to the subject M is changed as illustrated in FIG. 8. Referring to FIG. 8, it can be understood that the common area of each spectrum H, L is smaller than such area in FIG. 7 due to the operation of the dual filter Fs. In such way, the dual filter Fs are installed in order to provide an absolutely different quality of X-ray irradiated when two images are imaged.

### Confirmation Window for Switching Filter

Here, the inventor sets forth the switching confirmation window A installed to the holder 25. Referring to FIG. 2, the switching confirmation window A is installed near the hole 25b for mounting the combined filter and is a smaller hole than the hole 25b, and is installed in order to know the switching timing of the filter. The switching confirmation window A is installed as penetrating the holder 25, which is like a disk as well as the hole 25b for mounting the dual filter. Referring to FIG. 9A, only the switching confirmation window A is extracted and illustrated. Referring to FIG. 9A, the switching confirmation windows A are arranged on the virtual disk VA having the center axis 25a as the center thereof. The hole 25ba and the confirmation window A are not making a continuous hole.

The switching confirmation window A plays a role to inform the revolving status of the holder 25 to the switching detection element 20 that detects the switch of the single filter. Referring to FIG. 9B, the switching detection element 20 comprises a light emitting element 20a that emits the visible light and a light detection element 20b that detects the visible light. The light emitting element 20a and the light detection element 20 are aligned as sandwiching the holder 25. The switching detection element 20 detects the switching statue of the single filter based on whether the visible light emitted to the disk-like holder 25 in the penetrating direction passes through the switching confirmation window A installed to the holder 25 or not.

The inventor sets forth the reason why the switching confirmation window A is mandatory. Certainly, the holder revolving mechanism 17 is structured with e.g., a stepping motor so that an operation can be brought in reality as if the holder 25 is revolved exactly with only the predetermined angle. However, the moment when the filter is switched cannot be comprehended only with the holder revolving mechanism 17 and the control system therefor. Specifically, the switching detection element 20 and the switching confirmation window A are installed in order to actually measure the time when the combined are switched. The holder 25 has the holes 25b to which four filters F, Fs can be inserted. Accordingly, the switching confirmation window A is needed one for each of 4 filters.

Referring to the left side of FIG. 9, the switching confirmation window A corresponding to the dual filter Fs for subtraction is divided two parts, i.e., one for the switching confirmation window AH of the high voltage filter and another one for the switching confirmation window AL of the low voltage filter. Specifically, the switching confirmation window A corresponding to the dual filter Fs is structured as if the switching confirmation window AH of the high voltage filter and the switching confirmation window AL of the low voltage filter, and a channel element partitioning such confirmation windows AH, AL are aligned in the revolving direction of the holder 25. Further, the channel element is a portion that is intentionally left when the confirmation window AH, AL are formed by die-cutting the holder 25.

The high voltage filter switching confirmation window AH allows the visible light to pass only when the positional relationship between the holder 25 and the X-ray tube 3 is switched to the high voltage filter FH. Also, the low voltage filter switching confirmation window AL allows the visible light to pass only when the positional relationship between the holder 25 and the X-ray tube 3 is switched to the low voltage filter FL. According to such structure, the time when the dual filter through which the X-ray passes is switched to the high voltage filter FH when the dual filter Fs is being used and the time when switched to the low voltage filter FL can be discriminatingly and actually measured.

The inventor sets forth the aspect of that the switching times of the dual filter relative to the dual filter Fs is actually measured. Referring to FIG. 10, the state right after the dual filter, through which the X-ray passes, is switched to the high voltage filter FH is illustrated. At this time, relative to the passing region P of the X-ray on the dual filter Fs, the entire region thereof is positioned in the high voltage filter FH of the dual filter Fs. At this time, the visible light emitted from the light emission element 20a can be detected by the light detection element 20b after passing the high voltage switching confirmation window AH. A switching detection element 20 detects the fact in which the dual filter, which the X-ray passes through, is switched to the high voltage filter based on such detected results.

Referring to FIG. 11, the state in which the holder 25 is being revolved to switch the dual filter, through which the X-ray passes, from the state referring to FIG. 10 to the low voltage filter FL is illustrated. At this time, the passing region P of the X-ray on the dual filter Fs the entire region thereof is bridging the high voltage filter FH and the low voltage filter FL. At this time, the visible light emitted from the light emission element 20a can be detected by the light detection element 20b after passing the high voltage switching confirmation window AH. A switching detection element 20 that structurally detects to which the dual filter is switched either the high voltage filter or the low voltage filter.

Referring to FIG. 12, the status right after the dual filter, through which the X-ray passes, is switched from the state referring to FIG. 10 to the low voltage filter FL is illustrated. At this time, relative to the passing region P of the X-ray on the dual filter Fs, the entire region thereof is positioned in the low voltage filter FL of the dual filter Fs. At this time, the visible light emitted from the light emission element 20a can be detected by the light detection element 20b after passing the low voltage switching confirmation window AL. A switching detection element 20 that structurally detects to which the dual filter, which the X-ray passes through, is switched to the low voltage filter FL based on such detected results.

Referring to FIG. 13, the state right after the dual filter, through which the X-ray passes, is switched from the status referring to FIG. 12 to the other single filter F (e.g., a single filter for a spot imaging) is illustrated. At this time, relative to the passing region P of the X-ray on the single filter F, the entire region thereof is positioned in the single filter F for the spot imaging. At this time, the visible light emitted from the light emission element 20a can be detected by the light detection element 20b after passing the confirmation window A corresponding the single filter for the spot imaging. A switching detection element 20 detects the fact in which the combined filter, which the X-ray passes through, is switched to such filter based on such detected results.

According to the explanation so far, it is remained as questionable that the visible light passes through commonly any confirmation windows A, so that each confirmation window cannot be discriminated from others and therefore even the time when the filter is switched is decided, which filter is switched at that time cannot be decided. The inventor further sets forth about such question. The approximate time when the holder revolving mechanism ends revolving thereof can be calculated. Accordingly, an expected time when being switched can be figured out before the switching operation of the filter is activated. The switching detection element 20 initiates to detect visible light just before the expected switching time so that the actual switching time when the filter is switched can be measured. The switching detection element 20 initiates to detect a switching of the filter from the predetermined time before the expected filter switching time provided by the holder revolving control element 18. Such expected filter switching time is the time preliminarily calculated from the angle with which the revolving control element 18 revolves the holder 25.

For example, given the holder 25 must turn 90° to switch the target filter, it is assumed that the holder revolving control element 18 can calculate an approximate required 500 ms to revolve the holder 25 thereby. At this time, the switching detection element 20 decides the time, when the penetrated visible light is detected at around 500 ms (e.g., in the range of 450 ms-550 ms) after the holder 25 starts revolving, as the target filter switching time.

### Relationship Between Light Intensity and Switching of Dual Filter

Next, the inventor set forth the relationship between intensity of visible light detected by the light detection element 20b and switch of the dual filter. Referring to FIG. 14, the brightness of the visible light detected by the light detection element 20b is in three statuses. Specifically, the light detection element 20b detects no visible light at all in the Status 1, the light detection element 20b initiates to detect gradually the visible light in the Status 2 and the light detection element 20b detected the entire visible light in the Status 3.

Referring to FIG. 14, a switch of such detection statuses corresponds to the switch of the status of the confirmation window A. Specifically, when the holder 25 moves from the status in which the visible light is completely blocked by the holder 25, a part of the visible light passes through the confirmation window A in due course of time. When the holder 25 shifts further from such status, all beams relative to the visible light reach to the confirmation window A and pass through the confirmation window A in due course of time. According to such statuses of the confirmation window A, the status of light detection shifts from the status 1 to the status 3 via the status 2.

When the visible light is completely blocked by the holder 25, the light detection takes the Status 1. At this time, the entire region of the X-ray passing region P is out of the single filter F corresponding to the confirmation window A. In due course of time, when a part of the visible light passes through the confirmation window A, the light detection is shifted to the Status 2. At this time, it is in the status in which the partial region of the X-ray passing region P overlaps the single filter F. And, at last, when the entire part of the visible light passes through the confirmation window A, the light detection is shifted to the Status 3. At this time, the entire region of the X-ray passing region P overlaps the single filter F.

When the status in which the entire region of the X-ray passing region P overlaps the single filter is obtained, it is deemed at last that the switching operation of the single filter is initiated and the single filter is switched. Accordingly, when the time of switch of the single filter is detected, the switching detection element 20 decides the time when the visible light having a predetermined strength in association with the Status 3 is detected based on the output from the light detection element 20b as the time when the filter is switched. Accordingly, the switching detection element 20 decides as the set status of the single filter is False (i.e., the single filer has not been switched), when the light detection status is either Status 1 or Status 2. Then, the switching detection element 20 decides as the set status of the single filter is True (i.e., the single filer has been switched), when the light detection status is the Status 3.

Further, the beam diameter of the visible light emitted from the light emission element 20a is adjusted as the entire region of the X-ray passing region P overlaps the single filter F when the entire visible light passes through the confirmation window A.

As set forth above, an single filter F on the holder and the confirmation window A corresponding thereto are set forth as an example, but the operation of the device of the present invention is the same as the operation of the high voltage filter FH and the high voltage switching confirmation window AH corresponding thereto and the operation of the device of the present invention is the same as the operation of the low voltage filter FL and the low voltage switching confirmation window AL corresponding thereto. Accordingly, for example, the switching detection element 20 decides as the set status of the high voltage filter FH is True (the dual filter has been switched), when the visible light, which is passing through the high voltage switching confirmation window AH, detection status is the Status 3.

### Subtraction Imaging Based on the Combined Filter Switching Detection

The inventor sets forth that the device according to the present invention is operative to provide the subtraction imaging based on the dual filter switching detection. Referring to FIG. 15, each timing as to of the set status switching of the dual filter and a variety of operations is illustrated. Meantime, hereinafter, the inventor sets forth as the subtraction imaging is executed by first performing imaging relative to the high voltage followed by imaging relative to the low voltage is being performed

The operator instructs to initiate the subtraction imaging through a operation panel 26. In the case referring to FIG. 15, the holder 25 is not revolving at the beginning of imaging and the the dual filter through which the X-ray passes is already switched to the high voltage filter FH, so that the detection status of the visible light is the status 3, referring to FIG. 14, as to the high voltage switching filter confirmation window AH. Accordingly, the switching detection element 20 sends the signal, indicating that the dual filter applied for imaging right after the operator instructs is switched to the high voltage FH, to the X-ray control element 6. The X-ray control element 6 receives the above signal and realizes that the high voltage filter FH can be applied for imaging in association with the high voltage and instructs the X-ray tube 3 to irradiate X-ray in association with a high voltage condition. Following such instruction, the X-ray tube 3 irradiates X-ray by raising the voltage of the X-ray tube voltage.

When the X-ray radiation from the X-ray tube 3 ends, the X-ray tube control element 6 sends the signal indicating the end of radiation to the holder revolving control element 18. The holder revolving control element 18 initiates the holder to revolve via the revolving mechanism 17. In such way, the holder revolving control element 18 receives the X-ray output signal indication whether the X-ray is output or not from the X-ray tube control element 6 so that the holder 25 is initiated to shift from the time when the X-ray output ends in association with an imaging under the first high voltage condition. Followingly, the dual filter applied for imaging is switched from the high voltage filter FH to the low voltage filter FL. However, such switching requires a time in some length. On the other hand, the X-ray tube control element 6 holds and waits while nothing to do after the signal is sent.

When the holder 25 revolves, the detection status of the light detection element 20b relative to the switching detection element 20 varies. The detection status of the visible light becomes the Status 3 referring to FIG. 14 in due course of time via the status in which none of dual filter is set. At this time, the switching detection element 20 sends the signal, indicating that the dual filter applied for imaging is switched to the low voltage filter FL, to the X-ray control element 6.

The X-ray control element 6 receives the above signal and realizes that the low voltage filter FL can be applied for imaging in association with the low voltage and instructs the X-ray tube 3 to irradiate X-ray in association with a low voltage condition. Following such instruction, the X-ray tube 3 irradiates X-ray by lowering the voltage of the X-ray tube electric voltage. In such way, the switching detection element 20 sends the signal, indicating that the dual filter is switched due to the shift of the holder 25, to the X-ray tube control element 6 and then after receiving the signal relative to the switch of the dual filter, the X-ray tube control element 6 instructs the X-ray tube 3 to output the X-ray for second imaging under the low voltage condition.

Further, referring to FIG. 15, the time point ta is the time point at which the X-ray irradiation is initiated relative to the second imaging under the low voltage condition. When the holder 25 is revolved, inertia acts on the holder 25 so as to revolve continuously. Accordingly, even if the holder revolving control element 18 notifies the holder revolving mechanism 17 to suspend revolving of the holder 25, the revolving of the holder 25 cannot be stopped immediately. According to the aspect of the present invention, an imaging under the low voltage condition can be initiated from the time point ta before revolving of the holder 25 stops completely. Because when the visible light detection status turns to the Status 3 referring to FIG. 14 relative to the low voltage filter switching confirmation window AL, it is guaranteed that the dual filter applied for imaging is switched to the low voltage filter FL regardless whether the holder 25 is revolving or not.

The system according to the aspect of the present invention does not measure actually the end of the revolving of the holder 25 to decide switching of the dual filter. If the aspect of the present invention is based on such structure, the X-ray irradiation cannot be executed until the holder 25 completely stops to revolve According to the actual apparatus, the switching of the dual filter is completed even before the holder starts to revolve, so that it is not required to wait for stopping the holder 25 According to the aspect of the present invention, a switching of the dual filter is decided structure-wise based on whether the visible light passes through confirmation window AL, AH or not so that the X-ray irradiation can be executed before the holder 25 completely stops to revolve. Therefore, the continuous imaging speed can be increased.

The imaging under the high voltage condition or the low voltage condition can be executed, accordingly. In any imaging, the X-ray transmitted the subject M is detected by a FPD 4 and the FPD 4 sends the detected signal to a image generation element 11. The image generation element 11 generates an image PH relative to a high voltage imaging and an image PL relative to a low voltage imaging, and sends such images to a subtraction image generation element 12.

The subtraction image generation element 12 generates a subtraction image s by executing the subtraction process between the image PH and the image PL. Accordingly, the subtraction image s in which a bone and so forth of the subject M is emphasized. The aspect of the subtraction process actually executed by the subtraction image generation element 12 can be changed corresponding to an imaging purpose.

### Other Structure of the Apparatus According to the Aspect of the Present Invention

The operation panel 26 (referring to FIG. 1) is installed for the operator to input an instruction as to the initiation of the subtraction image imaging. Further, the main control element 27 (referring to FIG. 1) is installed to comprehensively control each control element. The main control element 27 comprises a CPU and brings the X-ray control element 6 and each element 11, 12, 18 into reality by executing a variety of programs. While each element can be activated separately by an arithmetic device to run each element. A storage element 28 (referring to FIG. 1) stores all parameters as to apparatus control including an X-ray irradiation condition referred by the X-ray tube control element 6. The display element 29 is installed to display a subtraction image s.

As set forth above, the X-ray imaging apparatus 1 according to the aspect of the present invention comprises an X-ray dual filter switchable quickly in association with the imaging conditions so that the subtraction imaging can be executed further stress-free. Specifically, according to the structure of the present invention, the holder 25 begins to shift since when the X-ray output ends relative to the first imaging in association with the subtraction imaging and then once the filters FH, FL are switched by shifting the holder 25 and when switching of the filter is actually measured, the X-ray tube 3 outputs the X-ray in association with the second imaging. According to the present invention, the filter can be changed to the suitable filter for imaging prior to the first imaging differently from the conventional aspect set forth referring to FIG. 17. Therefore, according to the aspect of the present invention, the imaging can be conducted right after the operator instructs regardless the phase of the dual filter FH, FL.

Further, according to the structure of the present invention, the second imaging can be performed right after it is confirmed that the filters FH, FL are switched in fact. Therefore, according to the structure of the present invention, the rate change of the continuous imaging can be achieved further quickly. For example, according to the aspect of the present invention, when the continuous imaging speed is changed so as to be slower, the first imaging is first of all performed and then the second imaging can be delayed to begin as long as needed. Referring to FIG. 17, if the high voltage filter FH and the low voltage filter FL are configured to rotate, the rotation rate must be changed, so that even first imaging is not operable until such rate becomes stable. According to the aspect of the present invention, such limitation would not take place.

Further as set forth above, if the high voltage filter FH and the low voltage filter FL are arranged in the shifting direction of the holder 25, and thereby one dual filter for subtraction imaging is structured, switching of the dual filter between the high voltage filter FH and the low voltage filter FL can be executed with a slight shift of the holder.

And if switching of the high voltage filter FH is confirmed through the high voltage filter switching confirmation window AH, the time point at which the filter is switched can be more assuredly figured out. Such circumstance is also applicable to the low voltage filter FL.

The present invention, following the below alternative Embodiment, can be implemented.

(1) According to the aspect of the above Embodiment, an X-ray irradiation under a high voltage condition is executed prior to an X-ray irradiation under a low voltage condition to perform a subtraction imaging but the aspect of the present invention is not limited to such configuration. The present invention can be applied to the subtraction imaging in which an X-ray irradiation under a low voltage condition is executed prior to an X-ray irradiation under a high voltage condition Such imaging can be operative as well as the operation set forth referring to FIG. 15. Specifically, the X-ray tube control element 6 executes the X-ray irradiation under the low voltage condition after receiving the signal, detecting that the low voltage filter FL is set, from the switching detection element 20 and the holder revolving control element 18 initiates the holder 25 to revolve after receiving the signal, indicating that the irradiation of the X-ray is completed, from the X-ray tube control element 6. At last, the X-ray tube control element 6 executes the X-ray irradiation after receiving the signal, detecting that the high voltage filter FH is set, from the switching detection element 20.

(2) According to the aspect of the above Embodiment, one subtraction image s is generated, but the aspect of the present invention is not limited thereto. The subtraction image s can be continuously generated and a video based on such images can be generated. In such cases, a dual filter, through which the X-ray passes, can be switched alternately between the high voltage filter and the low voltage filter. And, following the switching of the dual filter, an irradiation under the high voltage condition and an irradiation under the low voltage condition are repeated to provide alternately an image PH relative to the high voltage and an image PL relative to the low voltage. The subtraction image generation element 12 generates a subtraction image s by executing the subtraction process between the updated image PH and the updated image PL.

Specifically, when the updated image PH(n) relative to n time imaging is generated, the subtraction image generation element 12 reads out the image PL(n−1) relative to the n−1 time imaging generated right before the image PH(n) from the storage element 28 and generates a subtraction image s using such images PH(n), PH(n−1). Also, when the updated image PL(n+1) relative to n+1 time imaging is generated, the subtraction image generation element 12 reads out the image PH(n) relative to the n time imaging generated right before the image PL(n+1) from the storage element 28 and generates a subtraction image s using such images PL(n+1), PH(n). The generated subtraction image s is processed to the frame of the video by the a video generation element 13 referring to FIG. 1 and the frames are connected in generation order to provide the video relative to the subtraction.

(3) According to the aspect of the above Embodiment, the light emission element 20a emits visible light, but the aspect of the present invention is not limited thereto. The light emission element 20a can emit infrared light other than visible light.

(4) According to the aspect of the above Embodiment, dual filter through which the X-ray passes are switched between the high voltage filter FH and the low voltage filter FL by revolving the holder 25, but the aspect of the present invention is not limited thereto. The switching of the dual filter can be executed by moving the holder 25 back-and-forth linearly.

(5) According to the aspect of the above Embodiment, switching of the dual filter is detected by the switching detection element 20 and the confirmation window A of the holder 25, but the aspect of the present invention is not limited thereto. The holder revolving mechanism 17 can be actually operated by a servomotor and the output of the servomotor can detect switching of the dual filter, and further the switching detection element 20 can be actually executed with an encoder.

### REFERENCE OF SIGN

3 X-ray tube (Radiation source)

6 X-ray tube control element (Radiation source control means)

17 Holder revolving mechanism (Holder shifting means)

18 Holder revolving control element (Holder shifting control means)

20 Switching detection element (Detection means)

25 Holder

AH High voltage filter switching confirmation window

AL Low voltage filter switching confirmation window

FH High voltage filter

FL Low voltage filter

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

What is claimed is:

1. A radiographic imaging apparatus, that images a subtraction image by imaging twice using respectively different conditions, comprises:
a radiation source that irradiates a radiation;
a radiation source control circuit that controls said radiation source so as to irradiate alternately the radiation in association with a high voltage and the radiation in association with a low voltage;
a high voltage filter passes the radiation in association with the high voltage condition;
a low voltage filter passes the radiation in association with the low voltage;
a holder supporting said high voltage filter and said low voltage filter in a combination filter;
a holder shifting means that combines and switches between said high voltage filter and said low voltage filter by shifting said holder relative to said radiation source;
a holder shifting control circuit that controls said holder shifting means; and
a detection circuit that detects a combination filter location of said holder, a passing of said radiation; and a switched condition of either said high voltage filter or said low voltage filter, wherein:
said holder shifting control circuit initiates to shift said holder from a first time when the radiation output in association with the first imaging ends by receiving the radiation output signal, indicating whether the radiation is being output or not, from said radiation source control means, and
said detection circuit detects that the combination filter is switched following said holder's shift and sends a signal indicating a switching to said radiation control circuit, and
said radiation source control circuit allows said radiation source to output the radiation in association with the second imaging following a receiving of a signal indicating that the dual filter has been switched.

2. The radiographic imaging apparatus, according to claim 1, wherein:
said high voltage filter and said low voltage filter are arranged in a shifting direction of said holder and construct one dual filter for subtraction imaging.

3. The radiographic imaging device, according to claim 2, wherein:
(A) said detection circuit further comprises:
a light emission means that emits a light and that is in-place as sandwiching said holder and a light detection circuit that detects the light, and
(B1) a high voltage filter switching confirmation window that is installed on the holder and allows the light to pass through only in a positional relationship of said holder when the holder shifting means switches to the high voltage filter and said radiation source.

4. The radiographic imaging device, according to Claim, wherein:
(A) said detection means further comprises:
a light emission means that is in-place as sandwiching said holder and a light detection circuit that detects the light, and
(B2) a low voltage filter switching confirmation window that is installed on the holder and allows the light to pass through only in a positional relationship of said holder when the holder shifting means switches to the low voltage filter and said radiation source.
